(19) United States
(12) Patent Application Publication
(10) Pub. No.: US 2009/0004045 A1
(43) Pub. Date: Jan. 1, 2009

(54) Water Treatment Process and Apparatus

(76) Inventor: Jeffrey Fletcher, Kelowna (CA)

Correspondence Address: THOMAS E. MALYSZKO, SUITE 700, 205 - 5 AVENUE, S.W., CALGARY, AB, T2P 2V7, CA

(21) Appl. No.: 12/138,558

(22) Filed: Jun. 13, 2008

(30) Foreign Application Priority Data
Jun. 28, 2007 (CA) 2592767

(51) Int. Cl. A61L 2/04

(52) U.S. Cl. 422/1; 210/184

(57) ABSTRACT

A water treatment process whereby heat is used and recaptured to pasteurize water and or sewage effluent.

Contaminated waters are partially treatable utilizing bacterial and chemical processes, however some bacteria (e.g. Cryptosporidium and Giardia) are resistant to chemicals. Hence, the present process raises the temperature of the water to any desired temperature and for any desired length of time that would destroy most pathogens and viruses, including Cryptosporidium and Giardia, without the use of chemicals.

### FIELD OF THE INVENTION

The present invention relates to pollution reduction, and more particularly to the treatment of contaminated waste waters and drinking water.

### BACKGROUND OF THE INVENTION

Planet Earth should be our most prized possession, however, each and every day our waters are being polluted with industrial wastes & sewage.

Cities and towns, when economically feasible, do employ primary and secondary waste water treatment methods, however, the effluent from these methods may still contain viruses and pathogens that contaminate surface or subsurface waters.

Specifically:

1. It is estimated that at least 10,000 people per day die from the most basic water-borne diseases.

2. Indian and Northern Affairs Canada have tested and confirmed that in excess of 100 water systems in Northern Canada have potential health and safety concerns.

3. BC Waste Water Association estimates that 20% of 250,000 on-site sewage and disposal systems in BC are failing.

4. It was discovered in 2003 that sewage contains and spreads the SARS and West Nile Virus.

5. A recent Canadian health report claims “Cancer” rates are soaring to a devastating epidemic which will strike 41% of Canadian males and 38% of Canadian females. Studies have shown that chemicals like chlorine which are used to disinfect water interact with organic materials to form carcinogenic chemicals know as Trihalomethanes.

6. The US Environmental Protection Agency has recently identified more than 200 disinfection by-products from: Chlorine, Ozone, Chlorine Dioxide and Chloramines that may pose health risks.

7. In BC, there are at least 100 “Boil Advisories” any day of the year.

What is therefore desired is a novel water treatment apparatus and process which overcomes the limitations and disadvantages of the existing processes.

### SUMMARY OF THE PRESENT INVENTION

According to the present invention, there is provided in one aspect a process for economically pasteurizing sewage effluent, drinking water or any waters that require the absence of living organisms.

In another aspect the invention provides an apparatus whereby the desired contaminated water is pre-filtered and raised to an adequate temperature that will destroy most pathogens and viruses.

In another aspect the invention provides an apparatus whereby the operating temperature can be fluctuated at no additional cost for heat.

In yet another aspect the invention provides an apparatus that can utilize natural gas, propane, electricity, solar power or waste heat from other processes to provide the heat required, for instance an additional 1.1% heat.

### BRIEF DESCRIPTION OF THE DRAWING FIGURES

Embodiments of the invention will now be described, by way of example only, with reference to the accompanying drawings, wherein:

FIG. 1 is a schematic view of the apparatus and process according to a preferred embodiment of the present invention.

### DESCRIPTION OF PREFERRED EMBODIMENT

The invention described herein includes a process and an apparatus to utilize heat, recover and re-use heat to pasteurize fluids such as sewage effluent and contaminated waters to a high degree of purity.

It is an objective of this invention to achieve the aforementioned treatment with heat and without the use of chemicals, thereby eliminating chemical pollution and carcinogenic by-products.

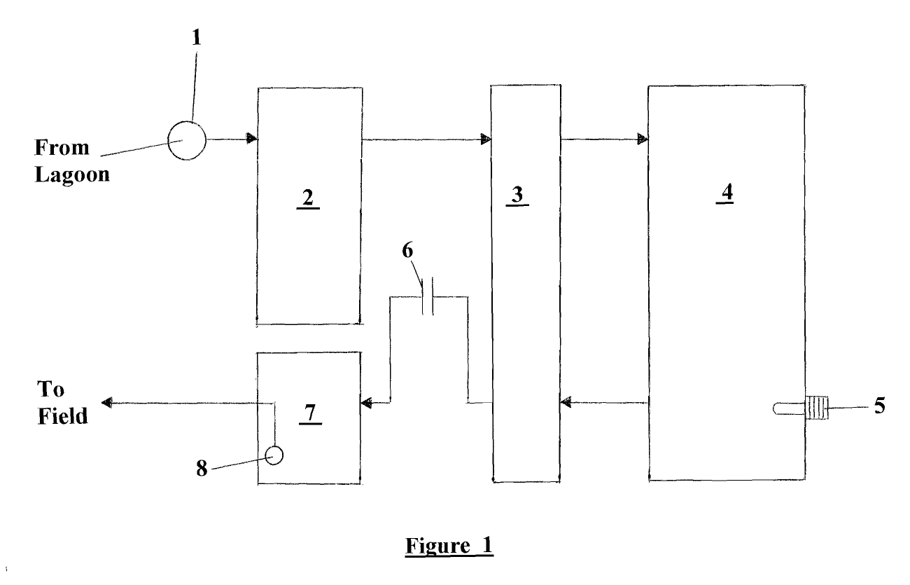

With reference to FIG. 1, the liquid stream to be pasteurized is forced through a “Filter” (#2), by “Pump” (#1), then through a “Heat Exchanger” (#3) with a 2 degree Fahrenheit approach, then into a “Volume Tank” (#4) with a “Heat Source” (#5). The “Heat Source” can be electrical, natural gas, propane, solar or any exterior heat source, such as waste heat from different processes. The “Volume Tank” (#4) is sized to accommodate any desired retention time. A different approach may be provided to suit specific circumstances. However, changing the approach (e.g. to 3 degrees F.) will effect the operating and capital costs of the apparatus and process.

The liquid then flows from the Tank (#4) back through the Heat Exchanger giving up approximately 98.9% of its enthalpy to pre-heat the off-setting inlet stream. The heat source provides the additional 1.1% required heat. It is understood that efficiencies of heat exchangers may vary, thus affecting the amount of additional heat required.

The liquid then flows through a “Siphon Breaker” (#6) and then on to an “Accumulation Tank” (#7) or directly to a Sub-Surface Disposal Field or to a Receiving Body of Water. “Pump” (#8) is optional depending on the aforementioned desires of disposal.

The above description is intended in an illustrative rather than a restrictive sense, and variations to the specific configurations described may be apparent to skilled persons in adapting the present invention to other specific applications. Such variations are intended to form part of the present invention insofar as they are within the spirit and scope of the claims below.

I claim:

1. A process for economically pasteurizing sewage effluent, drinking water or any fluids that require the absence of living organisms.

2. An apparatus comprising a means of pre-filtering a contaminated fluid and raising the fluid to an adequate temperature that will destroy most pathogens and viruses.

3. The apparatus of claim 2 wherein the operating temperature can be fluctuated at no additional cost for heat.

4. The apparatus of claim 3 comprising utilizing one of natural gas, propane, electricity, solar power or waste heat from other processes to provide the additional heat required.

5. The apparatus of claim 2 wherein the temperature is adequate for pasteurizing the fluid.
